(19) United States
(12) Patent Application Publication
(10) Pub. No.: US 2024/0189566 A1
(43) Pub. Date: Jun. 13, 2024

(54) VENT DEVICE, PORT ASSEMBLY AND VASCULAR ACCESS ASSEMBLY

(71) Applicant: B. Braun Melsungen AG, Melsungen (DE)

(72) Inventors: Eng Keat Ong, Penang (MY); Hui Kuun Teoh, Penang (MY); Jia Choon Law, Penang (MY); Mohd Zairizal Zakaria, Penang (MY); Muhamad Hazmi Ab Rahman, Alor Setar, Kedah (MY); Teng Sun Teoh, Penang (MY); Yie Min Lee, Penang (MY)

(73) Assignee: B. Braun Melsungen AG, Melsungen (DE)

(21) Appl. No.: 18/553,697

(22) PCT Filed: Apr. 5, 2022
(86) PCT No.: PCT/EP2022/059019

§ 371 Date: Oct. 2, 2023

Related U.S. Application Data
(60) Provisional application No. 63/171,230, filed on Apr. 6, 2021.

(51) Int. Cl. A61M 39/02; A61M 39/10

(52) U.S. Cl. CPC A61M 39/0247; A61M 39/10; A61M 2039/1077

(57) ABSTRACT

Intravenous sets, port assemblies, and vascular access assemblies can include a vent device for venting gas trapped therein. The vent device can connect to a connector, such as a needleless port having a valve, and the connector connected to an adapter. The vent device can have a valve opener, an engagement body, and a gas-permeable vent incorporated with the valve opener. In use, the valve opener is moveable from an initial non-venting condition, in which the valve opener does not actuate the valve and is held in-register with the valve by the engagement body, to a venting condition, in which the valve opener is actuated to open the valve to allow gas to pass from within the system the connector is connected to then flow out through the gas-permeable vent.

### TECHNICAL FIELD

The present invention relates, in general terms, to a vent device for venting gas from a line, for example, via an adapter. In some cases, the present invention can be used to vent gas from a peripheral intravenous catheter, or other vascular access devices and assemblies.

### BACKGROUND

A venous air embolism occurs when one or more bubbles enter a vein and restrict blood supply to particular human organs such as the heart, a lung, the brain, and so on. Arterial air embolisms are the same event, though occurring in an artery.

Air embolisms can cause a heart attack, stroke, respiratory failure, and can be fatal especially in neonatal patients. It can take as little as approximately 0.02 mL of air to cause a neonatal patient to suffer from ischemia or a restriction in blood supply to tissue.

Air embolisms can occur as a result of compressive injury, lung trauma, and compression, such as through scuba diving, and also through injections and surgical procedures. These incidents are common during brain surgery and infusion therapy where air trapped in the infusion line is not properly evacuated before drug infusion.

Currently, medical practitioners are trained to identify an air embolism and remove it. This is a responsive action rather than a pre-emptive action and therefore undesirable. Practitioners can expel air from the infusion line using a syringe. Practitioners can also aspirate trapped air by connecting the syringe to a needleless connector. This requires the practitioner to identify the trapped air and then remove it.

Both of the above remedies require a practitioner to identify an issue and resolve it, rather than removing or reducing the likelihood of the issue occurring.

Current air aspiration techniques result in additional time due to additional steps needing to be performed. This is undesirable in emergency situations where practitioners may already be distracted by other symptoms, procedures, and treatments. Moreover, there is additional waste and cost resulting from syringe use and the practitioner's time.

It would be desirable to provide useful, and preferably beneficial, alternatives.

### SUMMARY

Disclosed herein is a vent device for venting gas from a connector of an adapter or attached to the adapter, the connector comprising a valve, the vent device comprising:
a valve opener;
an engagement body; and
a gas-permeable vent at least in the valve opener,
wherein, in use, the valve opener is moveable from an initial, non-venting condition in which the valve opener is not in the valve and is held in-register with the valve by the engagement body, to a venting condition in which the valve opener opens the valve and gas can pass from within the adapter through the gas-permeable vent.

The gas-permeable vent may comprise a semipermeable material. The gas-permeable vent may comprise a flow path at least in the valve opener, the semipermeable material being located at a proximal end of the flow path.

The engagement body may engage the connector in both the initial, non-venting condition and the venting condition of the valve opener. The engagement body may extend distally of a distal end of the valve opener.

The engagement body may maintain a gap between the distal end of the valve opener and the valve when the valve opener is in the initial, non-venting condition. The engagement body and valve opener may be adapted to engage in fixed relation when the valve opener reaches the venting condition.

The engagement body may comprise a distal section for engaging the connector, a proximal section for supporting the valve opener, and a flexible intermediate section between the distal section and proximal section, the flexible intermediate section deforming so the valve opener can move to the venting condition.

The engagement body may move along the connector in a distal direction as the valve opener moves to the venting condition. The engagement body may comprise an engagement section having a distal region and a proximal region, the distal region being adapted to deform as the valve opener moves to the venting condition.

The engagement body may comprise at least one arm having a distal tip adapted to engage behind a narrowing in an outer surface of the connector to maintain the valve opener in the venting condition. The at least one arm may comprise a plurality of resilient arms adapted to engage behind the narrowing in the outer surface of the connector, in space relation. The plurality of resilient arms may comprise two arms adapted to engage behind the narrowing in the outer surface of the connector on opposite sides of the connector.

A distal end of the valve opener may be slotted.

The engagement body may rotate onto the connector to engage the connector, the engagement body comprising one or more finger grip members on an outer surface of the engagement body.

Also disclosed herein is a port assembly for a vascular access assembly, comprising:
an adapter comprising at least two connectors, for fluid connection of the connectors with a tube; and
a vent device as described above, attached to a first connector of said connectors.

The adapter may be a Y-port.

The vent device and first connector may define a sterile volume containing a distal end of the valve opener and a proximal end of the valve.

Also disclosed herein is a vascular access assembly comprising:
a port assembly as described above;
a catheter assembly; and
an extension tube connecting the port assembly to the catheter assembly.

The vascular access assembly may further comprise a clamp on the extension tube.

A vent device for venting gas from a connector of an adapter. The connector can comprise a valve and the vent device can comprise: a valve opener having a tip; an engagement body retaining the valve opener; and a gas-permeable vent formed with the valve opener; the valve opener is moveable from an initial non-venting condition, in which the valve opener is held in-register with the valve by the engagement body and the tip is not pushed against the valve to open the valve, to a venting condition in which the tip of the valve opener deflects the valve to open the valve and gas passes from within the adapter through the gas-permeable vent of the valve opener. The gas-permeable vent can comprise a semipermeable material.

The semipermeable material can be wedged inside a lumen of the valve opener.

The valve opener has a body and the gas-permeable vent can be a flow path formed through the body of the valve opener.

The semipermeable material can be located within the flow path of the body.

The engagement body can engage the connector in both the initial non-venting condition and the venting condition of the valve opener.

The engagement body can have a distal edge that extends distally of a distal end of the valve opener in the initial non-venting condition but not in the venting condition.

The engagement body can maintain a gap between the distal end of the valve opener and the valve when the valve opener is in the initial non-venting condition.

The engagement body and the valve opener can be axially fixed relative to one another in both the initial non-venting condition and the venting condition of the valve opener.

The engagement body can comprise a distal section for engaging the connector, a proximal section for supporting the valve opener, and an intermediate section between the distal section and proximal section, and wherein the intermediate section can be flexible and deformable so the valve opener can move from the initial non-venting condition to the venting condition when the intermediate section is deformed.

The engagement body can be movable along the connector in a distal direction as the valve opener moves from the initial non-venting condition to the venting condition.

The engagement body can comprise an engagement section having a distal region and a proximal region, the distal region can have a structure that is deformable as the valve opener moves from the initial non-venting condition to the venting condition.

The engagement body can comprise at least one arm having a distal tip adapted to engage behind a narrowing in an outer surface of the connector to maintain the valve opener in the venting condition.

The at least one arm can comprise a plurality of resilient arms adapted to engage behind the narrowing in the outer surface of the connector, in space relation.

The plurality of resilient arms can comprise two arms adapted to engage behind the narrowing in the outer surface of the connector on opposite sides of the connector.

The distal end of the valve opener can be slotted. There can be more than one slot at the distal end of the valve opener. For example, there can be three slots equally spaced at the distal end of the valve opener.

The engagement body can rotate onto the connector to engage the connector. The engagement body can comprise one or more finger grip members on an outer surface of the engagement body.

A port assembly for a vascular access assembly can comprise an adapter comprising at least two connectors; and a vent device can attach to a first connector of said at least two connectors.

The adapter can be a Y-site or a Y-port.

The vent device and the first connector can define a sterile volume containing a distal end of the valve opener and a proximal end of the valve.

A vascular access assembly can include a port assembly with an adapter comprising at least two connectors; and a vent device can attach to a first connector of said at least two connectors. An extension tube can connect the port assembly to a catheter assembly comprising a catheter hub and a catheter tube.

A clamp can be provided on the extension tube. A drip chamber and a spike can be fluidly connected to the extension tube.

The engagement body can include two laterally extending tabs to facilitate threading.

The valve opener can have a proximal flange having a dimension larger than an outer diameter of the engagement body. The valve opener can have a passage extending through the body of the valve opener and through the proximal flange.

The valve opener can have a locking body attached to the proximal flange and a tip connected to the locking body. A shoulder can be located between the tip and the locking body. The tip can be a male Luer.

The body of the valve opener can have a first set of notch and a second set of notch spaced from the first set of notch.

The first and second set of notches can each comprise two notches diametrically disposed about the lengthwise axis of the valve opener.

The engagement body can have a cantilever with an enlarged tip. The enlarged tip can engage the first set of notch in an initial non-venting condition of the valve opener. The enlarged tip can engage the second set of notch in a venting condition of the valve opener.

The valve opener can comprise a plurality of apertures and a lengthwise lumen, and wherein the plurality of apertures are in fluid communication with the lengthwise lumen.

The engagement body can comprise at least one window and at least one of the apertures of the valve opener aligned or exposed to the at least one window.

The engagement body can have a flexible section. The flexible section can be a living hinge, a wall surface with a thinned wall section, or a pliable or elastic material located between two harder material sections. The flexible section can deform to move the valve opener relative to a distal edge of the engagement body.

In an example, the valve opener can comprise a cantilever having an enlarged end and the engagement body can comprise an aperture formed on the wall of the housing. The enlarged end of the cantilever of the valve opener can engage the aperture on the engagement body.

The engagement body can have a proximal aperture and a distal aperture. The enlarged end of the cantilever on the valve opener can engage the proximal aperture in an initial non-venting condition of the valve opener and engage the distal aperture in the venting condition of the valve opener.

A method of venting gas entrapped in a connector. The method can comprise: connecting an engagement body of a vent device to a proximal end of the connector, the connector comprising a valve; aligning a tip of a valve opener secured to the engagement body in registration with the valve; and moving the valve opener after connecting the engagement body to the connector from an initial non-venting condition to a venting condition in which the tip pushes against the valve and gas entrapped in the connector flowing out through a gas-permeable vent formed with the valve opener.

Advantageously, the present invention enables gas, such as air bubbles, to be vented from vascular access assemblies.

Advantageously, embodiments of the vent device comprise a valve opener that does not come into contact with, or deform, a valve with which it is intended to be used, until the vent device is actively activated to move the valve opener to the venting condition. This ensures the valve is not damaged while in storage through prolonged, maintained deformation of the valve. Such damage may result in leakage through the valve. Therefore, for such embodiments, active activation is preferred over passive activation of the vent device. The valve can be part of a needleless port. The valve can be a piston that is deformable by the tip of the valve opener. The valve can be biases by a spring into a closed position.

Advantageously, embodiments of the vent device can be attached to, or engage, the connector prior to the valve opener coming into contact with the valve. This provides multiple advantages including the above-mentioned avoidance of deformation of the valve while the vascular access assembly incorporating the vent device is in storage.

Advantageously, embodiments of the invention can be deactivated after use by disengaging the engagement body from the connector. This enables the valve to return to its previous, closed condition.

As used herein, the term “valve” refers to a device that can be opened to facilitate passage of gas and/or fluid from one side of the valve to the other. In some devices, the valve may be referred to as a “septum”, “one-way valve”, “two-way valve” and similar, depending on the dimensions, application and other characteristics of the device incorporating the valve. In some devices, the valve is part of needleless port or connector.

As used herein, the term “gas permeable”, and similar terms such as “semipermeable”, refers to a particular material, structure, or component (e.g., the gas permeable vent) admitting the passage of gas therethrough. The term “semipermeable” refers to a particular material, structure, or component that allows gas to pass but prevents the passage of fluid. For example, air from within an adapter (e.g., a Y-port) may penetrate through or permeate a gas permeable device or membrane, or semipermeable device or membrane, but blood or saline solution from within the adapter may not. Semipermeable gas-permeable in this context may refer to a material being hydrophobic and therefore repelling fluids but not gases, or by having a pore size that is sufficiently large to permit the passage of gas therethrough but small enough to prevent the passage of fluid molecules.

The term “non-venting condition” refers to a condition of the valve opener in which it has not opened the valve and therefore is not venting gas from behind the valve. In the context of a port assembly, the non-venting condition applies up to the point at which gas can escape from a valve or a connector of the adapter, through the vent device. In some contexts, the “non-venting condition” may also encompass the valve opener being positioned entirely proximally of the valve, and may be in touch contact with the valve or spaced therefrom in a proximal direction. Conversely, the “venting condition” is a condition in which gas present in an adapter can pass from the adapter through the gas permeable vent.

### BRIEF DESCRIPTION OF THE DRAWINGS

Embodiments of the present invention will now be described, by way of non-limiting example, with reference to the drawings in which:

FIG. 1 provides a vascular access assembly comprising a vent device, in accordance with present teachings;

FIG. 2 shows a port assembly after detachment of the vent device;

FIGS. 3a and 3b show a vent device in accordance with the present teachings in a pre-activation/un-activated condition and an activated condition, respectively;

FIGS. 4 and 5 show the vent device of FIGS. 3a and 3b, in engagement with a connector, in the pre-activation/un-activated condition and the activated condition, respectively;

FIGS. 6a and 6b show a valve opener of a vent device in isometric and isometric cross-sectional views, respectively;

FIG. 7 shows components of a vent device in accordance with present teachings in disassembled and assembled states;

FIG. 8 shows an alternative embodiment of a vent device in accordance with present teachings;

FIGS. 9a and 9b show the disassembled components of the vent device of FIG. 8;

FIG. 10 illustrates the vent device of FIG. 8 in a cross-sectional, assembled state;

FIG. 11 shows the vent device of FIG. 10 in engagement with a connector, in an activated condition;

FIGS. 12 and 13 provide embodiments of an engagement body for use in a vent device in accordance with present teachings, showing inter alia different configurations of finger grips;

FIGS. 14, 15 and 16 illustrate the operation of the locking mechanism of the vent device of FIGS. 3a and 3b;

FIGS. 17, 18, 19, 20 and 21 illustrate the operation of an alternative locking mechanism for a vent device in accordance with present teachings;

FIGS. 22 and 23 show isometric and cross-sectional side views of a valve opener in accordance with present teachings;

FIGS. 24 and 25 show alternative embodiments of a gas permeable vent in accordance with present teachings;

FIGS. 26, 27 and 28 shows side, side cross-sectional and isometric views, respectively, of an alternative vent device in accordance with present teachings;

FIGS. 29, 30 and 31 show progressive steps in the use of the vent device of FIG. 26;

FIGS. 32a, 32b and 32c show progressive steps in the use of another embodiment of a vent device in accordance with present teachings;

FIGS. 33a and 33b show side and side cross-sectional views, respectively, of an alternative embodiment of a vent device in accordance with present teachings, in the pre-activation/un-activated condition;

FIGS. 34a and 34b shows side and cross-sectional views, respectively, of the vent device of FIGS. 33a and 33b in an activated condition;

FIG. 35 shows the vent device of FIG. 33b, and an activated condition when secured or engaged to a connector; and

FIG. 36 illustrates the vent device with an engagement body similar to that shown in FIG. 12, connected to an intravenous administration set (IV set).

### DETAILED DESCRIPTION

The detailed description set forth below in connection with the appended drawings is intended as a description of the presently preferred embodiments of vent devices provided in accordance with aspects of the present devices, systems, and methods and is not intended to represent the only forms in which the present device, system, and method may be constructed or utilized. The description sets forth the features and the steps for constructing and using the embodiments of the present device, system, and method in connection with the illustrated embodiments. It is to be understood, however, that the same or equivalent functions and structures may be accomplished by different embodiments that are also intended to be encompassed within the spirit and scope of the present disclosure. As denoted elsewhere herein, like element numbers are intended to indicate like or similar elements or features.

Vent devices, port assemblies, and vascular access assemblies as disclosed herein can engage a connector that includes a vent without coming into deforming contact with the vent. In this regard, the term “deforming contact” is intended to include contact under which the vent in the connector experiences deformation that can affect the operation or integrity of the vent if that contact is maintained. Without having a deforming contact, the vent device and the connector can be integrated in a packaging and stored as a unit. This enables the vent device to be stored as part of a port assembly or vascular access assembly without affecting operation of the connector to which the vent device is attached.

In the drawings and description hereafter, like reference numerals will be used to indicate identical functionally similar elements. It will be readily understood, particularly by virtue of the variations between the embodiments of the invention described herein, that the components of the invention as illustrated in the figures and described with reference thereto could be arranged and designed in a wide variety of different configurations without departing from the scope of the present teachings. Thus, the following detailed description, the accompanying figures, are intended to provide representative embodiments of the vent device, port assemblies and vascular access assemblies, without limiting the scope of the concepts taught herein.

Referring now to FIG. 1, a vascular access assembly (sometimes referred to as an extra vascular system or a vascular access system) 100 is used to communicate fluid to and from the vascular system of the patient. The assembly 100, when packaged, includes a needle for accessing the vasculature of the patient. The assembly 100 includes a catheter assembly 102 including an over-the-needle peripheral intravascular catheter tube hidden or located within the removable protective sleeve 104. The catheter tube can be made from polyurethane or other appropriate material and having a needle located in the lumen of the catheter tube and the needle attached to a needle hub 103. The catheter assembly 102 can have a catheter hub 105 with a side port and an integrated or attachable extension tubing 106 (in some instances this can also be referred to as a catheter) connecting the catheter assembly 102 to a port assembly 108. The port assembly 108 includes an adapter 110 and a vent device 112. Also, in the present embodiment, the vascular access assembly 100 comprises a tubing clamp 114 operatively located on the extension tube 106.

Other catheter assemblies can be used as needed with the present port assembly, and any adapter can be used that provides two or more connections, connectors or ports. Moreover, the vent device may be attached directly to the connector of the catheter assembly, such as when delivered in a product package and ready to use. Foreseeably, an adapter can be a Y-site with an integrated needleless valve integrated with one of the branches of the Y-site and the vent device of the present invention connected directly to the integrated needleless valve of the Y-site.

The vascular access assembly 100, or assembly for short, can protect a patient that the assembly is used with or used on from blood exposure during catheterisation. The assembly is configured to remove air from the port assembly and can protect patients from embolisms when use correctly.

In some exemplary embodiments of the vascular access assembly 100, a vent device 112 can pre-attach to a connector 116. For such embodiments, the assembly 100 can first be removed from a packaging. A pre-filled syringe can be removed from a different packaging, or optionally from the same packaging, and then attached to a port or connector 118 of the adapter 110 to which the vent device 112 is attached. The vent device 112 is then activated, such as by pushing on the activation part or valve opener located inside the housing of the vent device, as further discussed below, to open a flow passage for air to evacuate. The assembly 100 can then be flushed with the contents of the pre-filled syringe to expel air.

After removal of air from the assembly 100, a needle of the assembly can advance into the vasculature of the patient to establish vascular access. The catheter tube is then pushed over-the-needle into the vasculature and secured to the patient. The user then flushes the assembly 100 to create or allow flashback to confirm proper needle placement. Blood flashback can be confirmed with blood flowing through a notch in the needle, near the needle tip, and flowing into the annular space between the needle and the catheter tube, or blood can flow through the needle and into a flashback chamber of the needle hub, in combination with secondary flashback when blood flows between the needle and the catheter tube upon retraction of the needle tip proximal of the catheter tube opening. Upon confirmation of placement of the catheter tube within the vein, the catheter assembly is secured to the patient. The clamp 114 can then engage on the extension tube 106 to pinch the catheter tube and the pre-filled syringe can be separated and discarded. The vent device 112 can also be disengaged from the adapter 110.

In some cases, the needle may be retracted and the assembly 100 secured to the patient, before flashback confirmation. Moreover, other arrangements of steps can be used if the assembly 100 is not primed with saline solution prior to catheterisation.

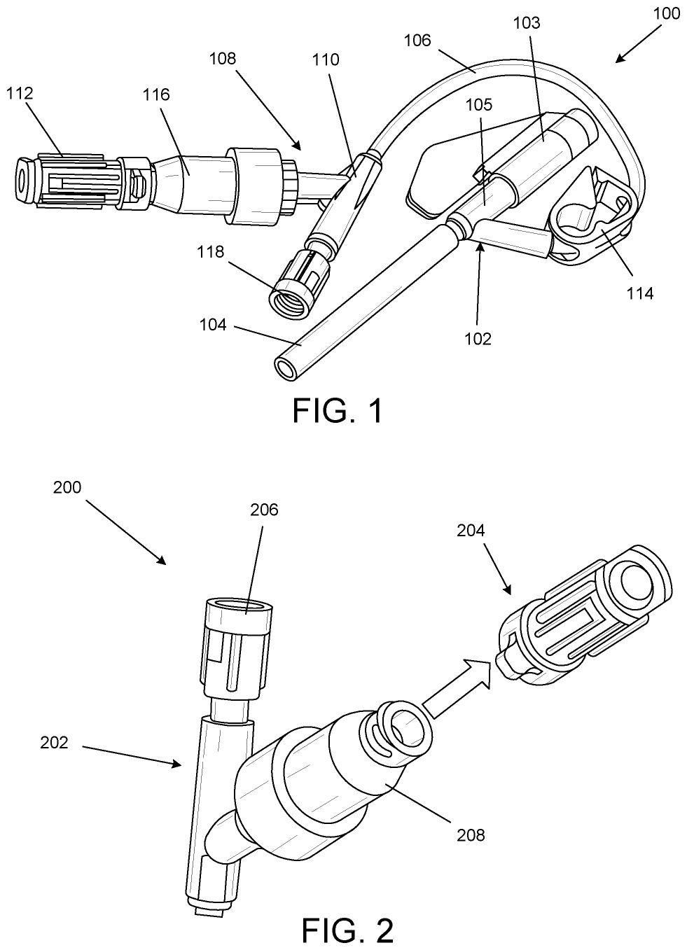

FIG. 2 shows a port assembly 200 comprising an adapter 202 and a vent device 204 that is detached from the adapter 202. The port assembly 200 may be used, for example, for or in a vascular access assembly, such as that shown in FIG. 1. The adapter 202 comprises two connectors 206, 208 and the adapter can fluidly connect the two connectors 206, 208 to a tube, such as the extension tube 106 of FIG. 1. The vent device 204 can attach to the connector 208 during use, by inserting the male tip of the vent device 204 to the female receiving end of the connector 208 and threading the vent device to the connector.

The adapter 202 has a body with a Y-port configuration. The port assembly 200 with the vent device 204 may be used in the same manner for adapters having more than two connectors. The connector 208 in the present embodiment includes a valve that can be opened by a valve opener of the vent device. In other words, the male tip of the vent device 204, when connected, can act as a valve opener to open the valve located inside the connector 208. In exemplary embodiments, the connector 208 can be a needleless connector and the valve located therein can be a collapsible piston, with or without a helical spring to facilitate return the collapsible piston after removal of the vent device from the connector. The other or second connector 206 of the port assembly 200 can also be a needleless connector or any other desired connectors.

In some embodiments, a vent device is preassembled with the adapter when provided in a sterile packaging. In another example, a port assembly comprising a vent device and an adapter are connected to a catheter assembly with an extension tube and preassembled as a vascular access assembly in a sterile packaging. As a result, when the port assembly 200 or vascular access assembly 100 (FIG. 1) is removed from a packaging, neither the proximal end or receiving end of the connector to which the vent device is attached, nor the valve opener of the vent device itself, will have had contact with the ambient environment or a user. The vent device and the connector therefore define a sterile volume containing a distal end of the vent device having a valve opener and a proximal end of the connector having a valve presented at the opening for pushing by the valve opener when actuated.

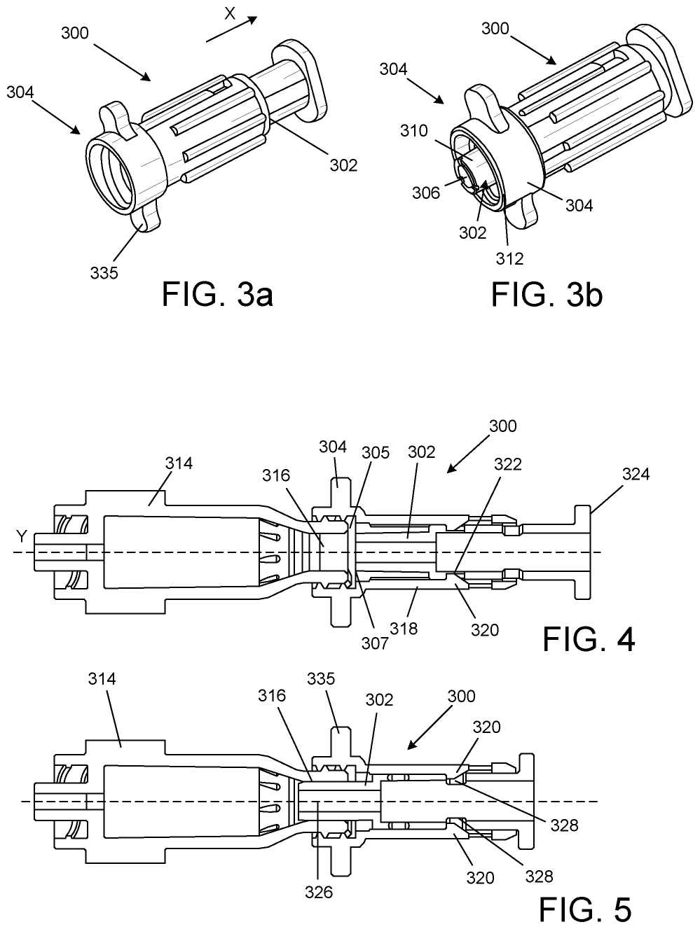

An exemplary vent device 300 is shown in FIGS. 3a and 3b. The vent device 300 is configured for venting gas from a connector comprising a valve in a port assembly. In an example, the vent device 300 includes a valve opener 302, an engagement body 304, and a gas-permeable vent 306. The engagement body may be considered a housing or a frame for containing or holding the valve opener. The engagement body has a further function of coupling to a port or connector, as further discussed below.

FIG. 3a shows the vent device 300 with the valve opener 302 in an initial or retracted position, which is a non-venting condition or first position. The valve opener 302 can have an elongated body that extends proximally in direction X, or is recessed inwardly into the body of the vent device relative to the engagement body 304 in the non-venting condition. The valve opener 302 can move from the first position or non-venting condition of FIG. 3a to a venting condition or second position shown in FIG. 3b. In the venting condition, the valve opener 302 moves distally relative to the engagement body 304, opposite direction X, and the tip of the valve opener is exposed at the distal end of the engagement body 304. A distal region or tip 310 of the valve opener 302 projects distally past the distal end edge 312 of the engagement body 304. While it is not essential that the tip 310 of the valve opener 302 projects distally of the distal end edge 312 of the engagement body 304, by projecting distally of the end edge 312 of the vent device 300, this ensures that a sufficient length of the valve opener 302, such as a sufficient length of the tip, projects into the connector (see, e.g., FIG. 5) to reliably open the valve to enable venting across the valve, as further discussed below.

With reference to FIG. 4, a cross-sectional side view of the vent device 300 of FIGS. 3a and 3b is shown attached to a needleless connector 314 in the vent device un-activated or pre-activation condition. FIG. 5 shows the vent device in an activated position. Refer initially to FIG. 4, the un-activated condition of the vent device 300 corresponds to the initial non-venting condition or first position of the valve opener 302. This un-activated or pre-activation condition is also the condition in which the vent device 300 is supplied in a product packaging. Accordingly, the engagement body 304 engages the proximal end of the connector 314 while the valve opener 302 is in the initial non-venting condition.

While in the un-activated condition of FIG. 4, the engagement body 304 holds the valve opener 302 in-register with a valve 316 of the connector 314. The “in-register” condition refers to the valve opener 302 and valve 316 being in alignment along a longitudinal axis, for example along axis Y. For example, the tip of the valve opener 302 is located adjacent the upper surface of the valve 316 and aligned to move against the valve so that distal movement of the tip of the valve opener can cause the tip to contact and push the valve 316 to open the valve.

Also shown in FIG. 4, the engagement body 304, when attached to the connector 314, can maintain a gap 305 between the distal end-most tip 307 of the valve opener 302 and the valve 316 when the valve opener is in the initial, non-venting condition. This gap 305 defines a sterile volume, particularly when the vent device 300 is provided pre-attached or preassembled in the port assembly.

The engagement body 304 engages the connector 314 by a threaded connection. As shown, the engagement body 304 of the vent device 300 has interior threads that thread with exterior threads of the housing of the connector 314. It will be understood that other connection mechanisms may be used as appropriate, such as a twist-lock connection, friction fit or other form of connection. Also shown at the distal end of the engagement body 304 are extending tabs 335, which are optional. The extending tabs 335, which can include two or a pair, can be incorporated to facilitate gripping and as leverage to thread and/or unthread the vent device 300 with the connector 314.

The vent device 300 is protected against activation by temporarily securing the valve opener 302 from moving, such as by using an engagement arrangement. In the present embodiment, the engagement arrangement comprises two cantilevers 318 incorporated on the engagement body 304. In an example, each cantilever 318 is provided as a prong or tine formed with three unconnected edges and one attached edge to the housing, as shown in FIG. 3b. Each cantilever 318 therefore has an elongated body and the attached edge allows the cantilever to act like a leaf spring that can flex about the attached edge. Each cantilever further has an enlarged tip 320 that sits, or at least partially projects, in a corresponding notch 322 in the valve opener 302. The engagement between the enlarged tips 320 and the notches 322 provide resistance against distal advancement of the valve opener 302. In an example, the two cantilevers 318 are diametrically opposed on the housing of the engagement body 304, but a non-symmetrical arrangement about the body 304 is contemplated provided the two cantilevers are aligned with the notches on the valve opener 302.

One or both of the notches 322 and the enlarged tips 320 may incorporate tapered surfaces to permit the two to slide relative to each other once sufficient force is actively applied in a distal direction at the proximal end 324 of the valve opener 302, which has an enlarged flange having an outer perimeter with a diameter that is larger than the main body of the vent device. The valve opener 302 is movable relative to the engagement body 304. When a sufficient distally directed force is applied to the flange at the proximal end 324, the force moves the valve opener 302 from the initial, non-venting condition to the venting condition as shown in FIG. 5. The venting condition of the valve opener 302 corresponds to the activated condition of the vent device 300. The distally directed force must overcome the engagement between the two enlarged tips 320 of the two cantilevers and the two notches 322 on the valve opener 302. In alternative embodiments, the valve opener 302 may incorporate the two cantilevers and the engagement body 304 may incorporate the two notches for receiving the two enlarged lips of the cantilevers.

In the venting condition of FIG. 5, the male tip of the valve opener 302 slides through the opening of the connector 314 to open the valve 316 so that gas can pass from within the port assembly, such as 108 and 200 of FIGS. 1 and 2, and can vent through flow channels opened up by the valve opener 302 pressing on the valve 316 and then through a gas-permeable vent 326 of the valve opener 302 of the vent device 300. Flow channels for gas flow can be formed on the valve, in the interior of the body of the connector, or both. When the valve 316 is pushed by the tip of the valve opener 302, the valve moves further into the interior cavity of the housing to open the flow channels. Moreover, when in the venting condition, the engagement body 304 and the valve opener 302 are adapted to engage in fixed relation. In an example, the fixed relation can be achieved by the engagement arrangement fixing the relative positions of the valve opener 302 and the engagement body 304. In the venting condition of FIG. 5, the flange at the proximal end prevents further advancement of the valve opener 302 in the distal direction, by providing a physical barrier that abuts the housing to prevent further travel. In a particular example, the enlarged tips 320 of the cantilevers snap into engagement with a second set of notches 328, located proximally of the first set of notches 322. In an example, the first set of notches 322 embody divots or recesses formed partially through the thickness of the valve opener 302. In an example, the second set of notches 328 are located proximally of the first set of notches 322 and are formed as divots or recesses with greater depths than the first set of notches, including being formed as through holes through the thickness of the valve opener 302. The engagement between the enlarged tips 320 and the second set of notches 328 in the venting condition is generally irreversible to maintain the valve opener 302 in the distally advanced position to open the valve 316. That is, the valve opener 302 cannot retract proximally relative to the engagement body 304 once the valve opener is in the venting condition. Although not desired, the user can manually slide a pick through the open edges of the two cantilevers to disengage the enlarged lips 320 from the second set of notches 328.

The gas-permeable vent 326 is located at least in part in the valve opener 302. As shown, the gas-permeable vent 326 is the lumen of the valve opener. For example, the valve opener 302 can comprise a body or structure and a lumen is formed through the body to define the gas-permeable vent. The gas-permeable vent 326 allows gas to pass from within the port assembly through the valve and out through the gas-permeable vent 326 to vent outside of the port assembly. In an example, the gas-permeable vent 326 is a passage through the valve opener 302 for directing gas within the system out through the vent device. In some examples, the gas-permeable vent 326 can include a hydrophobic filter that allows gas or air to pass but prevents water-based fluids from passing. In some cases, the gas can vent to the atmosphere and in other cases the gas can vent to an internal volume of the vent device. To that extent, the gas-permeable vent 326 is in the valve opener 302 so as to convey gas from the port assembly through the valve opener 302. In an example, a hydrophobic filter or membrane may be placed at the opening of the tip of the valve opener. In another example, the hydrophobic filter may be placed within the lumen at the tip of the valve opener. In other examples, the filter or membrane may be located nearer a proximal region of the gas-permeable vent. The gas-permeable vent may also extend into or through other components of the device.

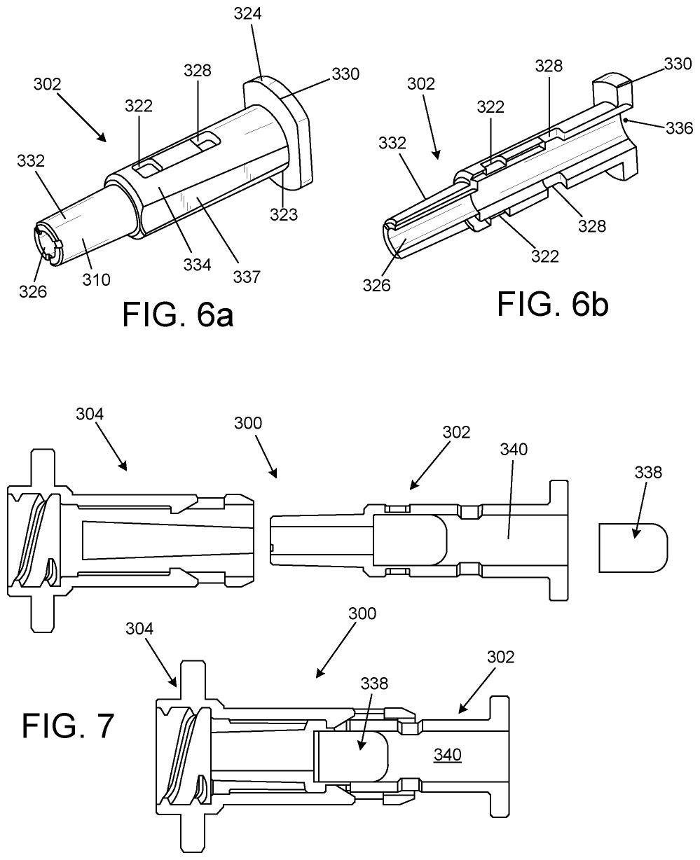

FIG. 6a shows the valve opener 302 in a perspective view and FIG. 6b shows the valve opener 302 in a cross-sectional perspective view. The valve opener 302 comprises an actuation portion 323 at the proximal end 324, which can be manipulated to activate the vent device. In an example, the actuation portion 323 comprises a radially extending flange 330. The radially extending flange 330 provides a surface that is sufficiently large to comfortably be actuated by the finger of a user (e.g., a physician or nurse). The valve opener also comprises an activation end 332 comprising a tip 310.

The activation end 332 with the tip 310 is adapted to be inserted into the connector to which the vent device is attached to activate the valve of the connector. The shape of the activation end may be selected depending on the type of valve the vent device is required to open. In the present embodiment, the tip 310 of the activation end 332 is a male Luer. The activation end 332 therefore tapers in a distal direction for fitting into the female Luer of the connector. However, where the device to be vented by the activation end 332 of the vent device is a septum, the activation end can embody a different activation mechanism, such as being a sharp spike or a hollow needle.

Between the activation end 332 and actuation portion 323 is a locking body 334. A shoulder or enlarger is located between the activation end 332 and the locking body 334. The locking body 334 comprises the first set of notches 322 and the second set of notches 328. Each pair of notches can be diametrically opposed about the locking body 334. In an example, a first notch 322 can align with a second notch 328 along a line that is parallel to the lengthwise axis of the valve opener 302. The first notch 322 can be located closest to the shoulder or enlarger while the second notch 328 can be located at about the mid-point of the locking body, between the proximal end where the flange 330 is located and the distal end of the locking body. The locking body 334 has a length that is longer than the length of the activation end 332, such as about 50% longer to about 300% longer. In an example, the locking body 334 is generally round along an end cross-section. In the present embodiment, the locking body has a round cross-section with two side truncated sections 337, which are optional. The truncated sections 337 on the body can eliminated air traps with the interior of the housing and allow for a smoother activation of the valve opener 202. Each the truncated section has a planar surface strip extending from the distal end to the proximal end. The planar surface strip can have a constant height. As shown, the planar surface strip 337 has a varying height from the distal end to the proximal end of the locking body 334. In an example, the housing or engagement body 304 has corresponding planar surfaces to cooperate with the truncated surfaces, to thereby prevent rotation of the valve opener when moving inside the engagement body.

The gas-permeable vent 326 extends through the length of the activation end 332, the locking body 334, and the actuation portion 323. The valve opener 302 therefore has body with a hollow interior and the gas-permeable vent 326 vents gas from within the connector to which the vent device is attached and out of a proximal end of the valve opener 302 through an aperture 336 at the proximal end 324.

FIG. 7 shows a cross-sectional exploded view of an alternative vent device 300 and an assembled cross-sectional view of the same vent device 300. The present vent device comprises a valve opener 302 and an engagement body 304, similar to the other vent devices discussed elsewhere herein. In the present embodiment, a semipermeable material 338 can be included. The semipermeable material 338 can be inserted into a conduit or lumen 340 of the valve opener 302, thereby forming a gas-permeable vent 326 with a semipermeable material 338. Semipermeable materials can include materials made from a mixed cellulose esters (MCE) membrane, an acrylic copolymer matrix, and polytetrafluoroethylene (PTFE) material, to name a few non-limiting examples. The semipermeable material 338 can embody a plug-like structure and can fit within the lumen 340 via frictional interference fit. Optionally a projection or tab can be provided in the lumen to physically retain the semipermeable material 338 therein.

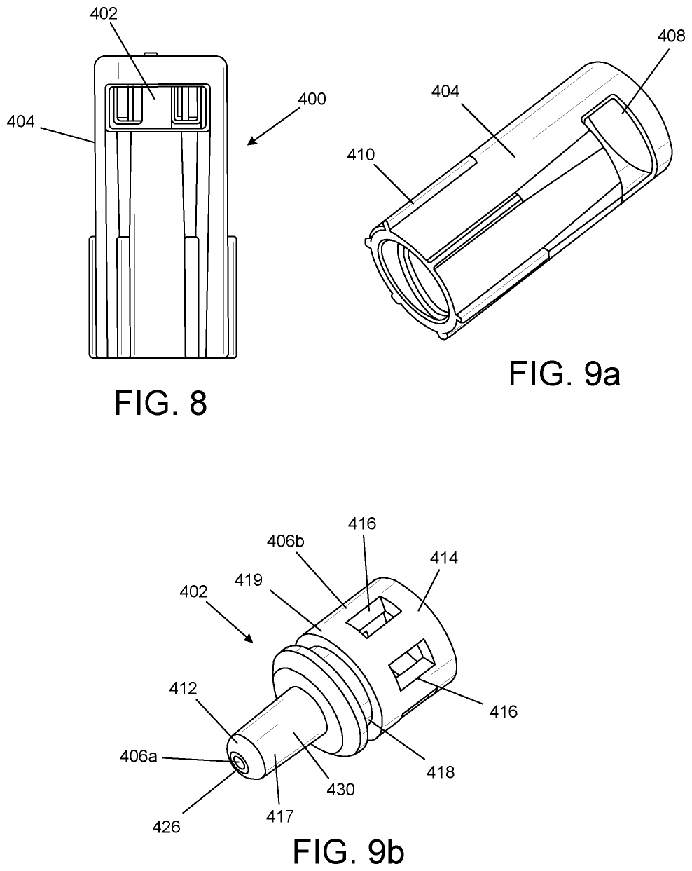

FIGS. 8 to 11 illustrate a further embodiment of a vent device 400 in accordance with further aspects of the invention, shown in various disassembled and assembled states. Like vent devices discussed elsewhere herein, the present vent device 400 is configured for venting gas from a connector of a port assembly, which can be part of a vascular access assembly. The vent device 400 includes a valve opener 402 and engagement body 404. FIG. 8 shows the vent device 400 in an assembled state while FIG. 9a shows the engagement body 404 separated from the valve opener 402, which is shown in FIG. 9b.

Refer initially to FIG. 9a, the engagement body 404 has a generally cylindrical hollow body and includes an aperture or window 408. A similar second aperture or window can also be provided on the opposite side of the engagement body 404. Together, the two windows 408 enable gas to vent from within the connector to which the vent device 400 is attached to atmosphere. Each of the two windows 408 can be generally rectangular and each can follow the contour or curvature of the engagement body. Each window can be formed as a through hole that has a through passage formed through the material layer or wall of the engagement body. Each window can have a height and a width. In an example, the width can be about 15% to 40% of the circumference of the engagement body 404.

The engagement body 404 also includes a plurality of finger grips 410 on an exterior thereof. Each of the finger grips 410 can comprise an elongated raised rib disposed in space relation around the circumference, or outer surface, of the engagement body 404 relative to the other finger grips. The finger grips 410 provide surfaces for fingers of a user to grip to rotate the vent device 400 during removal of the device from an adapter of a port assembly. Where the vent device 400 is supplied separately from the adapter, the finger grips 410 may also facilitate attachment of the vent device 400 to the adapter.

FIG. 9b shows a valve opener 402 with a gas-permeable vent comprising an inlet 406a at the distal end 412 of the tip 417 of the valve opener 402, and an outlet portion 406b in a proximal region 414 of the valve opener 402. The outlet portion 406b presently comprises a plurality of apertures 416. The apertures 416 communicate gas from within the valve opener 402 to atmosphere, or elsewhere outside of the valve opener 402. The apertures 416 can be spaced around the circumference of the proximal region or base section 419 of the valve opener 402. In an example, each aperture 416 is generally rectangular in shape and can be evenly spaced along the base section 419 of the valve opener 402. This ensures that, regardless of the orientation of the valve opener 402 in the engagement body 404 after assembly of the vent device 400, at some of the apertures 416 of the valve opener 402 will be in open communication with one of the two windows or apertures 408 of the engagement body 404 to enable venting from within the gas-permeable vent 426 through the apertures 416 of the valve opener and through the windows 408 of the engagement body 404.

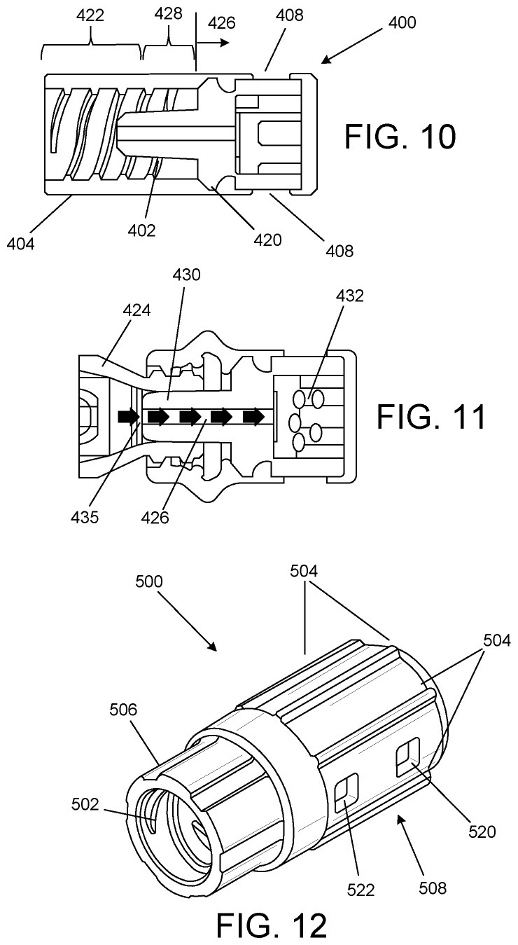

It will be appreciated that only a single finger grip 410 may be provided and/or a single aperture 416 may be provided, or any other number of finger grip and aperture as appropriate. In an example, the valve opener 402 comprises an engagement ring 418. The engagement ring 418 can be an engagement recess ring formed around the circumference of the base section 419, embodying a recess. The engagement ring 418 is located closer to the shoulder between the tip 417 and the base section 419 than to the proximal end edge of the base section. The engagement ring 418 is configured to engage a corresponding engagement projection 420 (see FIG. 10) formed with the engagement body 404. The engagement ring 418 can be considered an engagement first section and the engagement projection 420 can be considered an engagement second section, and wherein the engagement first section can engage the engagement second section, such as having a projection projecting into a recess. In an example, both the engagement ring 418 and the engagement projection 420 can be annular or has an annulus configuration. The engagement ring 418 and the engagement projection 420, when engaged, ensure that the valve opener 402 is maintained in a fixed axial relation within the engagement body 404. Accordingly, the valve opener 402 does not axially move, in use, relative to the engagement body 404. This engagement maintains alignment of the apertures 416 with the windows 408 to ensure that at least some of the apertures 416 are always in open communication with the windows 408 and no relative axial movement can cause the two to misalign.

FIG. 10 is a cross section of the assembled vent device 400 of FIG. 8 in a non-venting condition. The engagement body 404 comprises a distal section 422 having interior threads for receiving a male tip of a connector 424, a proximal section 426 for supporting the valve opener 402, and an intermediate section 428 between the distal section 422 and proximal section 426.

In an example, the intermediate section 428 is flexible or pliable. In a particular example, the intermediate section 428 can deform so the valve opener 402 can move from a non-venting condition to a venting condition, as shown in FIG. 11. The intermediate section 428 can buckle upon experiencing a compressive force, which can cause the wall surface of the intermediate section 428 to buckle outwardly away from the lengthwise axis of the engagement body 404 to shorten the length of the body to thereby effectively move the tip 417 at the distal region 430 of the valve opener 402 distally forward to open the valve 435 of the connector 424 connected to the threaded distal section 422 of the engagement body 404 (FIG. 11). Flow channels for gas flow can be formed on the valve, in the interior of the body of the connector, or both. Air may then pass from within the connector 424 along the flow channels and through the gas-permeable vent 426, into a chamber or lumen 432 of the gas-permeable vent 426. From there gas may pass out of the apertures 416 and out the windows 408 of the engagement body to the atmosphere. In other embodiments, the chamber or lumen 432 may retain the gas therein.

As reflected in FIG. 10, the distal section 422 is any section forward of the intermediate section 428 and is used for engaging the connector 424. The distal section 422 and the intermediate section 428 do not need to be contiguous, although they are shown as being contiguous. For example, an extension or length of the engagement body may be disposed between the distal section 422 and the intermediate section 428. Similarly, the proximal section 426 is shown as being any portion disposed proximally of the intermediate section 428 and is used for supporting the valve opener 402. Again, the proximal section 426 and the intermediate section 428 do not need to be contiguous but may instead have another section of the engagement body 404 disposed therebetween.

The flexible intermediate section 428 may have a living hinge that allows it to buckle, may be formed with a material composition that is different when compared with the distal section 422 and proximal section 426, may have a thin-wall section that can readily buckle, or any other appropriate arrangements that result in preferential deformation of the intermediate section 428 when compared with the distal section 422 and proximal section 426.

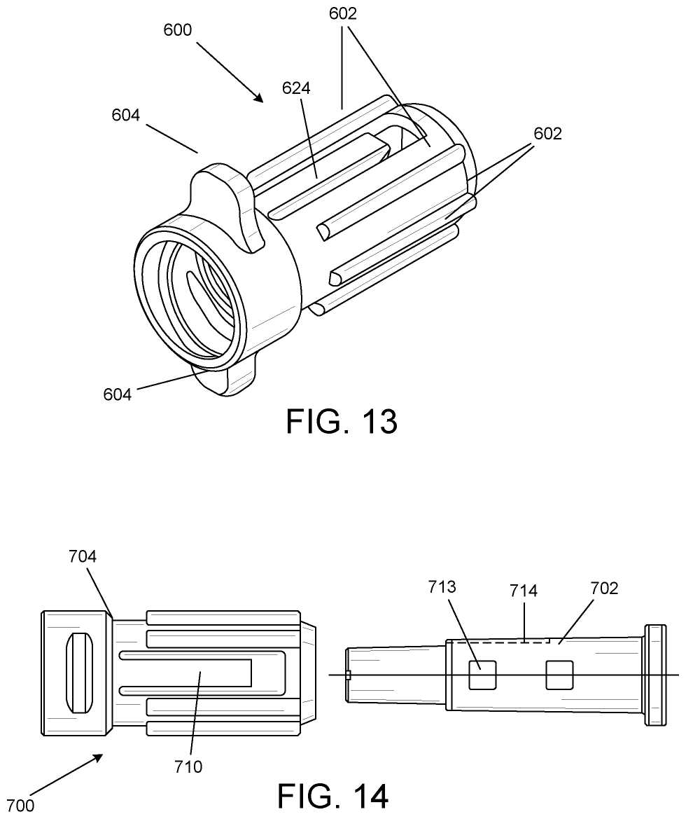

FIGS. 12 and 13 show two alternative embodiments of an engagement body for cooperating with a valve opener. The engagement body 500 of FIG. 12 is attachable to a connector having a valve. For example, the distal end of the body 500 can have internal threads at the threaded connection 502 for receiving external threads of the connector. The engagement body 500 can include a plurality of finger grips 504. The finger grips can be provided to facilitate control of the vent device, such as during threading and unthreading of the vent device and the connector. The finger grips 504 can be spaced around an outer surface of the body and comprises a raised projection in the radially outwardly direction relative to the central axis of the body. Also shown is a first set of engagement notch 520 and a second set of engagement notch 522, located distally of the first second of engagement notch. The two sets of notches 520, 522 can align along a line parallel to the lengthwise axis of the engagement body 500. Each of the two set of notches 520, 522 can comprise a pair of recesses or openings diametrically opposed about the body. Projections on a valve opener are configured to engage the first set of notches 520 in a non-venting condition and advanced distally to engage the second set of notches 522 in the venting condition.

The engagement body 500 can include a second engagement mechanism for engaging a connector. The second engagement mechanism 506 can be disposed on an external surface of the engagement body 500. The second engagement mechanism 506 can be inserted into a port of the connector and may connect, for example, by friction fit. It will be appreciated, that the engagement body in accordance with aspects of the invention may include one or more mechanisms for engaging a connector so that the engagement body may act or function as a universal housing for mating to or with multiple connector types having different connection requirements. For vent devices that are preassembled with adapters, only the engagement mechanism suitable for attaching to the relevant connector of the adapter needs to be provided. For vent devices that are provided separately from adapters, it may be advantageous to provide multiple engagement mechanisms to facilitate connection to a variety of connectors.

Engagement body 500 also includes apertures 508, such as first and second set of notches 520, 522, that form part of an engagement mechanism as discussed with reference to FIGS. 17 to 21, further discussed below.

FIG. 13 provides an engagement body 600 that is similar to the engagement body 300 of FIGS. 3a and 3b. A plurality of finger grips 602 in the form of raised ribs 602 are provided with the body as well as a pair of wings 604, also referred to as extending tabs. The wings 604 can be disposed equally distantly around a circumferential of the outer surface of the engagement body 600, near the distal end than the proximal end. That is to say, two wings or extending tabs 604 are disposed on diametrically opposite sides of the engagement body 600. It will be appreciated that various configurations of finger grips may be used, in various numbers from one or more, and in some embodiments, it may be suitable to provide no finger grips. The pair of wings 604 may be used as leverage to thread and unthread the engagement body to the connector. A pair of cantilevers 624 (only one shown) may be provided as an engagement mechanism to engage with corresponding notches on the valve opener, as previously discussed with reference to FIGS. 4 and 5.

FIGS. 14 to 16 show an alternative embodiment of a vent device 700 in accordance with further aspects of the invention. The vent device 700 includes a valve opener 702 and an engagement body 704 with a gas-permeable vent 706. The device 700 is consistent or similar with the vent device 300 discussed elsewhere herein.

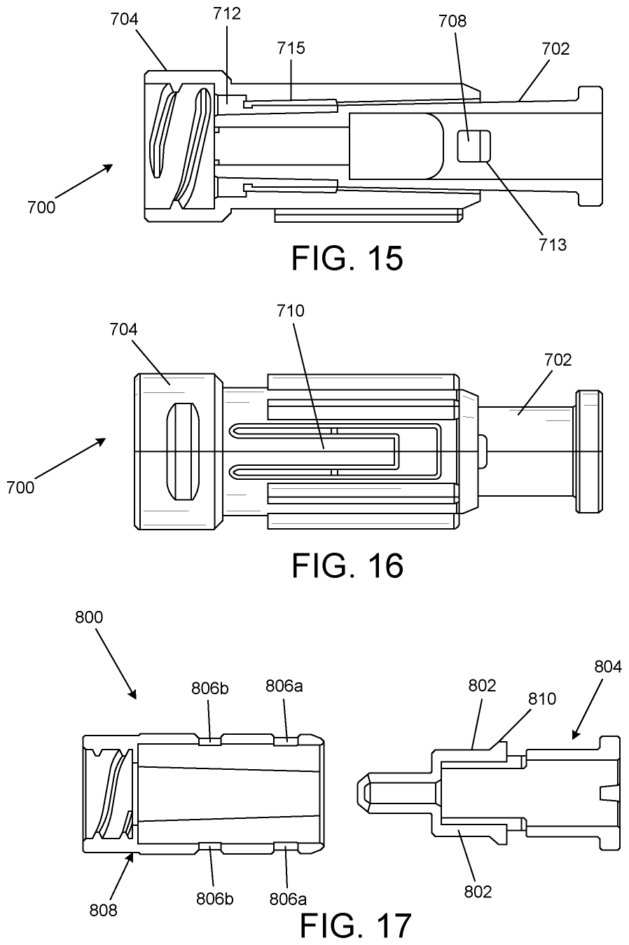

FIG. 14 shows the vent device 700 in an exploded view, with the valve opener 702 separated from the engagement body 704. FIG. 15 is a cross-section side view showing the vent device 700 in an assembled condition in which the enlarged end 708 of cantilever 710 of the engagement body 704 is received in an aperture 713 of the valve opener 702 in a non-venting condition. In a perspective view, the vent device comprises a pair of cantilevers each with an enlarged end 708 for engagement corresponding set of apertures 713.

The enlarged ends 708 of the two cantilevers 710 include an inclined surface and a flat surface Arranged or aligned to deflect or to resist when the valve opener is advanced in the distal direction. As the valve opener 702 moves in the distal direction towards the venting condition, such as when pressed at the flanged proximal end, a proximal edge of the apertures 713 slides along the inclined surface of the enlarged ends 708, enabling the cantilever 710 to bend outwardly away from the central lengthwise axis of the vent device. Conversely, if an attempt was made to pull the valve opener 702 from the non-venting condition in a proximal direction, the flat surface of the enlarged ends 708 would abut a an edge of apertures 713 and prevent movement of the valve opener 702 in a proximal direction relative to the engagement body 704.

The internal volume 712 of the engagement body 704 in which the valve opener 702 travels may be asymmetrical, with the valve opener 702 being similarly asymmetrical. This may guide the valve opener 702 longitudinally within the engagement body 704. Alternatively, as shown in FIG. 15, the engagement body 704 may include one or more internal guides 715 in internal volume 712 or interior cavity of the body. The guide 715 may be a protrusion, such as an elongated rib, or a recess that slides within a complementary opposing recess or protrusion 714 (FIG. 14) in the valve opener 702. The internal guides 715 may slide within the recess 714 until a proximal portion of the guide 715, which can have a stop, reaches a proximal portion of the recess 714 and the two cause a physical stop or abutment to prevent further distal movement of the valve opener 702 relative to the engagement body 704. It will be appreciated that various other mechanisms can be used to guide movement of the valve opener 702 in relation to the engagement body 704 to ensure respective portions of the locking mechanism on each of the engagement body 704 and valve opener 702 are aligned, and prevent rotation of the valve opener within the engagement body 704. FIG. 16 is a side elevation view of the vent device 700 of FIG. 15, in the assembled non-venting condition.

FIGS. 17 to 21 show an alternative embodiment of a vent device 800 in accordance with still further aspects of the invention. The vent device 800 of the present embodiment comprises an engagement body 808 and a valve opener 804, similar to other vent devices discussed elsewhere herein, with a few exceptions. In the present embodiment, the vent device 800 utilizes an engagement mechanism in which one or more cantilevers 802 are incorporated with the valve opener 804 rather than the engagement body 808. The engagement body 808 in the present embodiment are provided with complementary first and second set of apertures 806a, 806b for cooperating with the cantilevers 802 on the valve opener 804.

Similar to the operations of vent device with the configuration of cantilevers and apertures of the embodiment shown in FIGS. 4 and 5, the enlarged ends 810 of the cantilevers 802 (FIG. 17) are configured to project into and be received in the proximal pair of apertures or first set of apertures 806a of the engagement body 808 in the non-venting condition, when the valve opener 804 is assembled to the engagement body 808. Upon activation of the vent device 800, such as by applying a distally directed force at the flanged proximal end of the valve opener, the cantilevers 802 bend inwardly, aided by the tapered surfaces at the enlarged ends 810, so that the enlarged ends 810 disengage from the first set of apertures 806a and move along until the resiliencies of the two cantilever arms 802 cause the two enlarged ends 810 to project into the distal pair of apertures or second set of apertures 806b, where the valve opener 804 is moved to the venting condition.

The enlarged ends 810 include a flat surface and an inclined surface arranged or aligned to deflect or to resist when the valve opener is advanced in the distal direction. As the valve opener 804 moves in the distal direction towards the venting condition, a proximal edge of the apertures 806a slides along the inclined distal surface, enabling the cantilever 802 to deflect outwardly. Specifically, the inclined surface creates a pair of component forces when contacting the proximal edge thereby generating a pair of components forces that include a radial component that causes the cantilever to deflect. Conversely, if an attempt was made to pull the valve opener from the non-venting condition in a proximal direction, the flat proximal surface of the enlarged ends 810 would abut a proximal edge of apertures 806a and prevent movement of the valve opener 804 in a proximal direction relative to the engagement body 808. In this instance, no pair components forces are generated as the two flat surfaces create a physical abutment or stop.

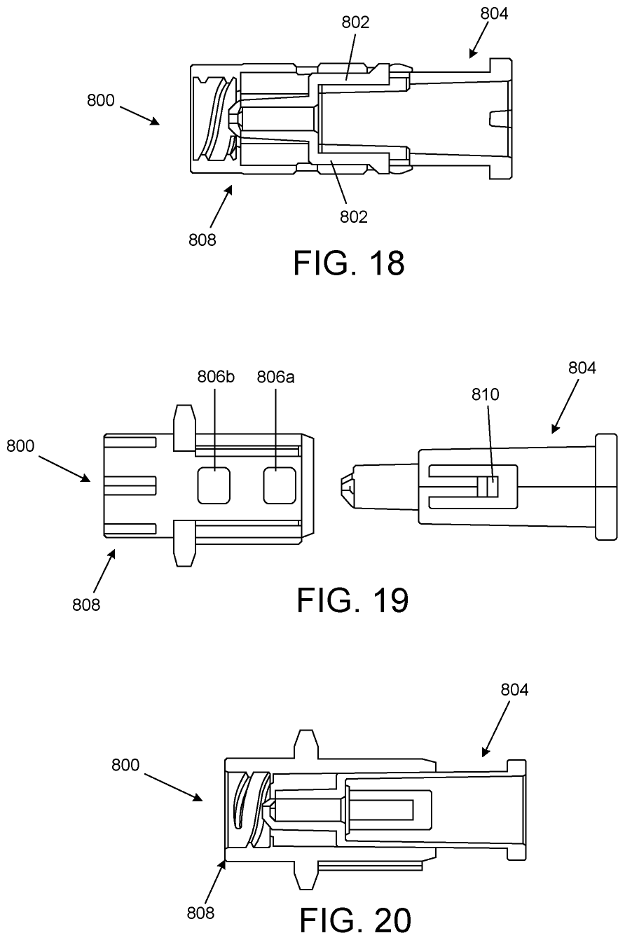

FIG. 18 shows vent device 800 in an assembled state and in a non-venting position.

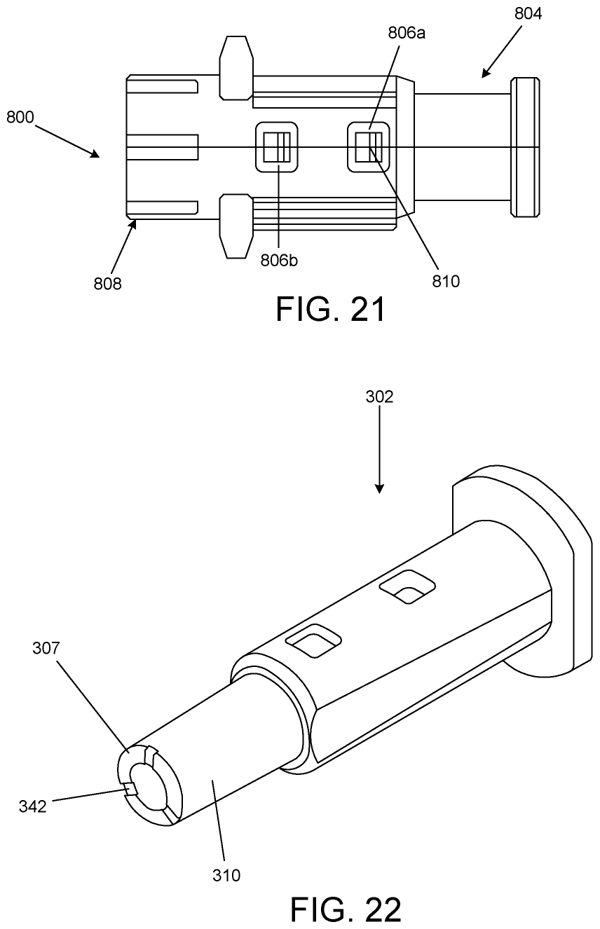

FIGS. 19 to 21 show the vent device 800 of FIGS. 17 and 18 rotated 90-degrees about the lengthwise axis. FIG. 19 shows the vent device in a plan exploded view, FIG. 20 shows the vent device in an assembled cross-section view, and FIG. 21 shows the vent device in a side plan view. These views more clearly demonstrate alignment between the enlarged ends 810 and the first and second set of apertures 806a and 806b.

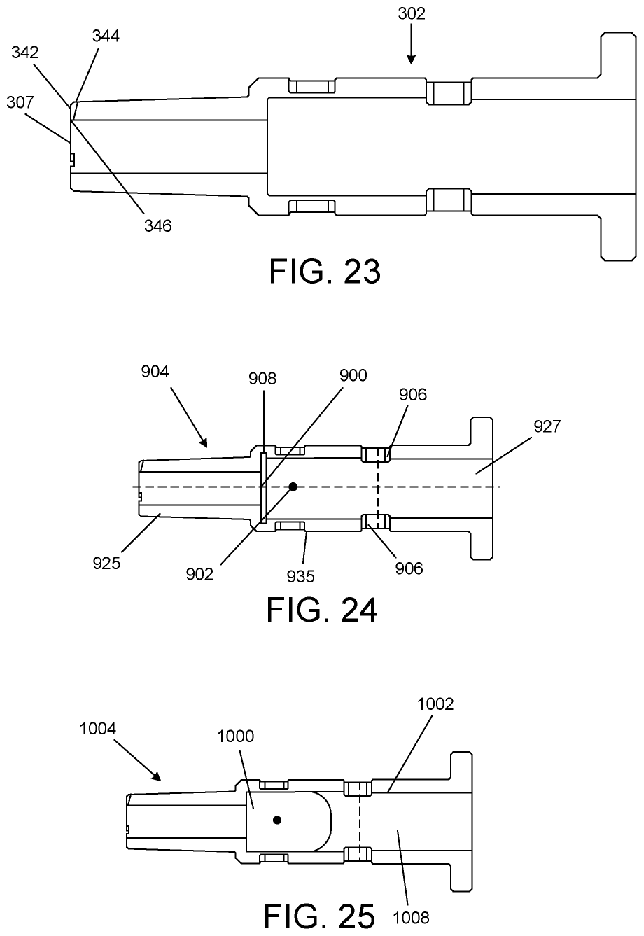

FIGS. 22 and 23 are perspective and side cross-section views of a valve opener 302 in accordance with aspects of the invention. The distal end 307 of the valve opener 302 is slotted or has slots 342. In this sense, one or more slots 342, and presently three slots 342, are formed at the distal end 307 edge of the tip. The slots 342 can function as channels to assist with the escape of air from within the connector, especially if the distal edge 307 is abutted against a flat surface, in which case air can pass between the flat surface and the slots 342. The slots 342 can also assist with the retention of blood internally of the Luer connector or valve to prevent blood exposure.

In an example, each slot 342 can taper inwardly in the radial direction such that the surface at the distal end 307 having the slot extends slightly further in a proximal direction at an outer edge 344 of the distal end than at an inner edge 346 of the distal end. In other examples, each notch as a generally U-shape with the length or height of the side edges of the U-shape slot being selectable to control the depth or size of the slot.

FIGS. 24 and 25 show alternative valve openers 904, 1004 in accordance with further aspects of the invention. Each valve opener comprises a gas-permeable vent configured to convey gas, such as air, out of a connector to which the vent device that the valve opener operates with is attached in the venting condition. As shown, each gas-permeable vent comprises a flow path and a gas-permeable or semipermeable material located inside the lumen thereof. The gas permeable or semipermeable material may be placed anywhere along the lumen of the valve opener. For example, the material may be placed at the distal-most end of the valve opener or at some location intermediate the distal end and proximal end of the valve opener.

In the embodiment shown in FIG. 24, the semipermeable material 900 is a semipermeable membrane located in the flow path or lumen 902 of the valve opener. Gas flowing through the tip 925 of the valve opener 904 passes through the membrane 900 and flows in the direction out the proximal opening 927. However, as gas travels along the path of least resistance, gas may also escape laterally through apertures 906 incorporated along the base section 935 of the valve opener 904. The membrane material 900 can attach in the flow path 902 by a peripheral recess 908 in the flow path 902, the material 900 projecting into the peripheral recess 908 and retained thereby.

The valve opener 1004 embodiment shown in FIG. 25 is similar to the valve opener of FIG. 24 with the exception that the gas-permeable membrane 1000 of the present embodiment resembles a bulk material or a plug. The bulk material or plug 1000 of the present membrane is fixed in position by friction fit against an internal surface 1002 of the valve opener 1004, in the flow path or lumen 1008. For example, the plug can be provided with a larger outer diameter than the internal diameter of the flow path to then be retained via compressive friction fit. Other attachment mechanisms, such as ultrasonic welding or adhesive, may be used as appropriate.

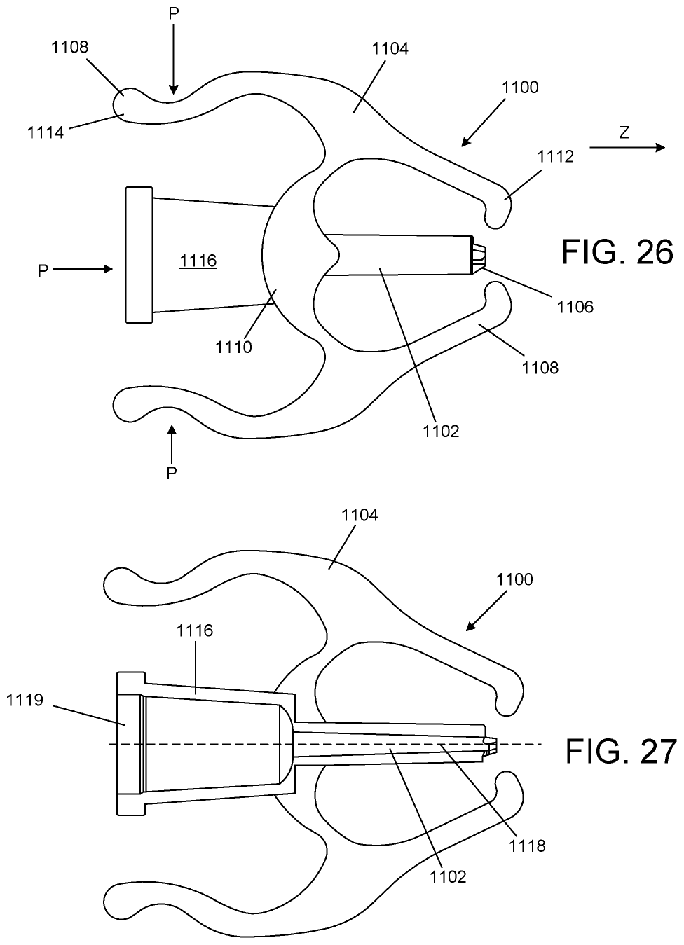

FIGS. 26 to 31 show an alternative embodiment of a vent device 1100 in accordance with further aspects of the invention. The present engagement body 1104 comprises a plurality of arms 1108 and a proximal section 1110 between the arms 1108, which functions as a bridge connecting the arms. Each arm has a distal tip 1112 to engage an outer surface of the connector to couple the vent device to the connector to then open the valve of the connector. The proximal section or bridge 1110 also secures the valve opener 1102 to the engagement body 1104. In an example, the bridge 1110 comprises an opening and a saddle for receiving the body of the valve opener 1102.

As with other vent devices disclosed herein, the vent device 1100 of the present embodiment is configured for one-handed use. To that end, the user or practitioner can move the vent device 1100 from a non-vent condition to an activated condition within just one hand or a few fingers of one hand. In an example, two fingers of a hand may apply laterally inward pressure P to the proximal portions 1114 of arms 1108 of the vent device 1100 while a third finger applies a distally directed force to the proximal end of the enlarged proximal protrusion 1116 of the valve opener. The forces applied by the three fingers of the user are shown in the directions of the arrows marked with the letter P.

In some configurations, the vent device 1100 may comprise only a single arm or some other arrangement of arms. In the present embodiment, the engagement body and the valve opener are axially fixed relative to one another in both the initial non-venting condition and the venting condition of the valve opener.

FIG. 27 shows a cross-sectional view of the vent device 1100 of FIG. 26. The gas-permeable vent 1118 extends through the valve opener 1102. The flow path of the gas-permeable vent 1118 can include a gas-permeable or semipermeable material identified by reference 1119, which can be a semipermeable membrane. Notably, as is evident in the cross-section view of FIG. 27, the enlarged proximal protrusion 1116 may embody a female Luer and configured to connect to a male Luer connector or other connectors.

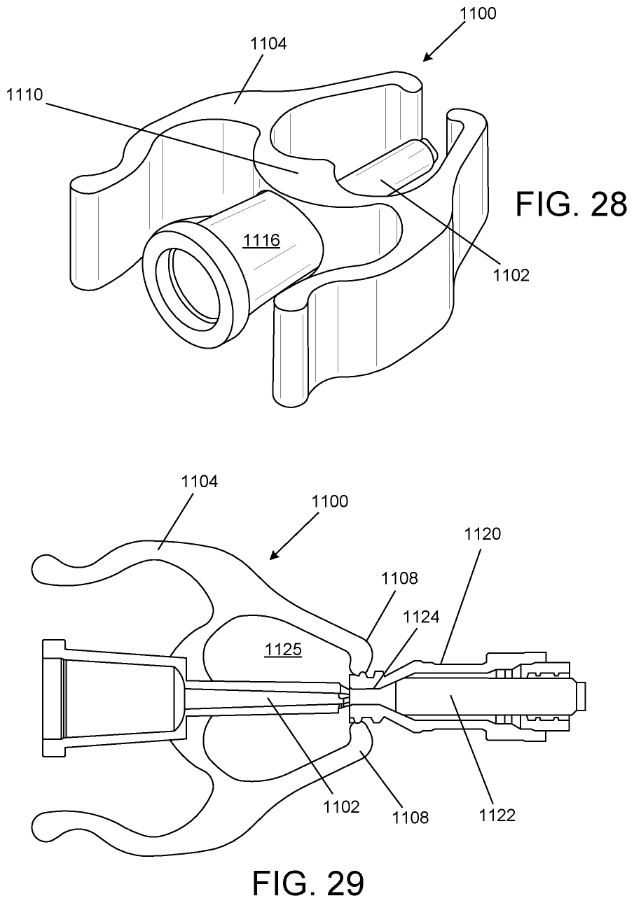

FIG. 28 shows a respective view of the vent device 1100 of FIGS. 26 and 27 showing that the proximal section or bridge 1110 supporting the valve opener 1102 either directly or by supporting the enlarged proximal protrusion 1116 of the valve opener that is integral with or otherwise connected in fixed relation to one another. For example, the bridge 1110 can have an opening with a complementary receiving recess for receiving the body of the connector. The two can be secured via friction fit, snap fit, detents, adhesive, welding, or combinations thereof.

FIG. 29 shows the vent device 1100 of FIGS. 26-28 connected to a connector 1120 in the pre-activation or un-activated condition, in which the tip 1125 of the valve connector has not activated the valve inside the connector. The shape of the vent device is such that the distal end portions of the plurality of resilient arms 1108 of the engagement body 1104 are located slightly distally of the distal end of the tip 1125 of the valve opener 1102. The engagement body 1104 therefore can grip the connector and support the valve opener 1102 in alignment with the connector 1120. If air 1122 is trapped within the connector 1120, air can be vented through the valve opener 1102 when the valve opener is actuated to open the valve 1124 and create a gas flow path through the valve opener 1102.

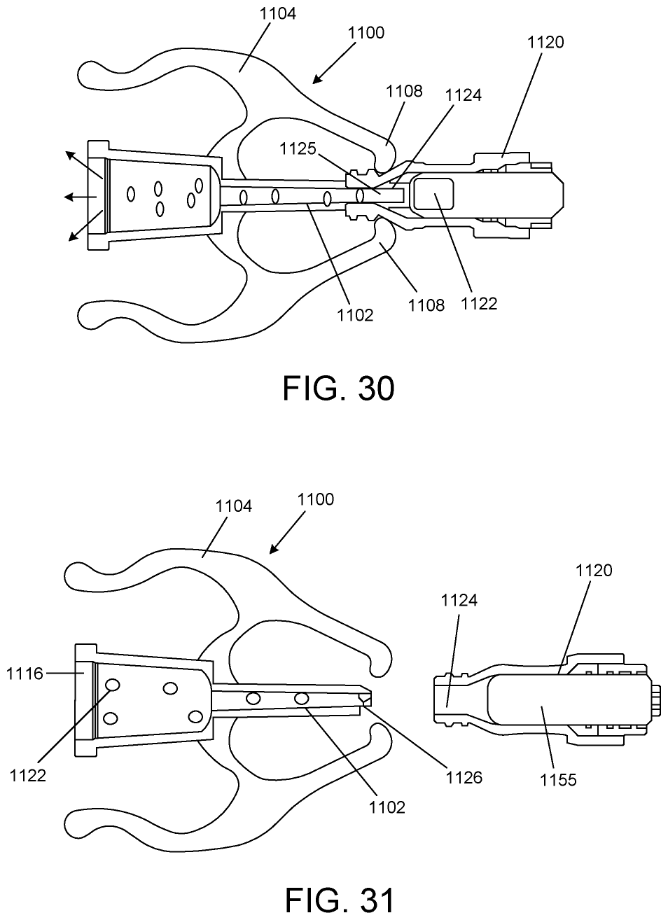

FIG. 30 shows the vent device 1100 of FIG. 29 in an activated condition corresponding to the venting condition of the valve opener 1102. In the activated condition, the tip 1125 of the valve opener 1102 has entered the connector and opened the valve 1124 of the connector 1120, such as pushing the valve in the distal direction or compressing the valve to one the one or more flow passages, as previously discussed. When the valve 1124 is opened, gas or air 1122 from within the connector 1120 can exit through one or more of the flow passages through the connector and enter the valve opener 1102. While in this vent condition, the resilient arms 1108 engage behind a narrowing section of the neck of the connector in the outer surface of the connector. In an example, the narrowing section is provided by a trough in a screw thread on the outer surface of the connector. Therefore, while the proximal portions of the resilient arms 1108 may be squeezed inwardly to increase the distance between the distal tips of the resilient arms 1108 to move the vent device to the activated condition, the vent device 1100 may instead be rotated into position and the arms 1108 may therefore be rigid or fixed. To that end, the distal end of one of the arms may be slightly longer than the distal end of another one or more of the arms to enable the arms to travel along the screw thread while maintaining alignment of the valve opener 1102 with the connector 1120, to better follow the helix pattern of the exterior threads.

The arms 1108 engage the connector 1120 in spaced relation. In an example, the two arms 1108 of the vent device 1100 are disposed on opposite sides of the connector 1120.

FIG. 31 shows the vent device 1100 separated from the connector 1120. Notably, the gas 1122 previously located in the connector 1120 is now entirely vented from the connector 1120 and into the vent device. The venting is aided by flushing the connector 1120 with fluid, which is now full of fluid 1155. Additionally, some of the fluid 1155 has passed through the valve 1124 and into the vent device 1100 but is unable to pass from the vent device the atmosphere due to the hydrophobic membrane. For example, passage to the atmosphere is prevented by the semipermeable material 1116. Moreover, the distal end 1126 of the tip may be configured with a device for preventing spillage back out the opening. In an example, the distal end 1126 may include a one-way valve, a constriction, or other devices for preventing fluid, such as blood, from dripping out of the opening at the distal end 1126, after disengagement of the vent device 1100 from the connector 1120.

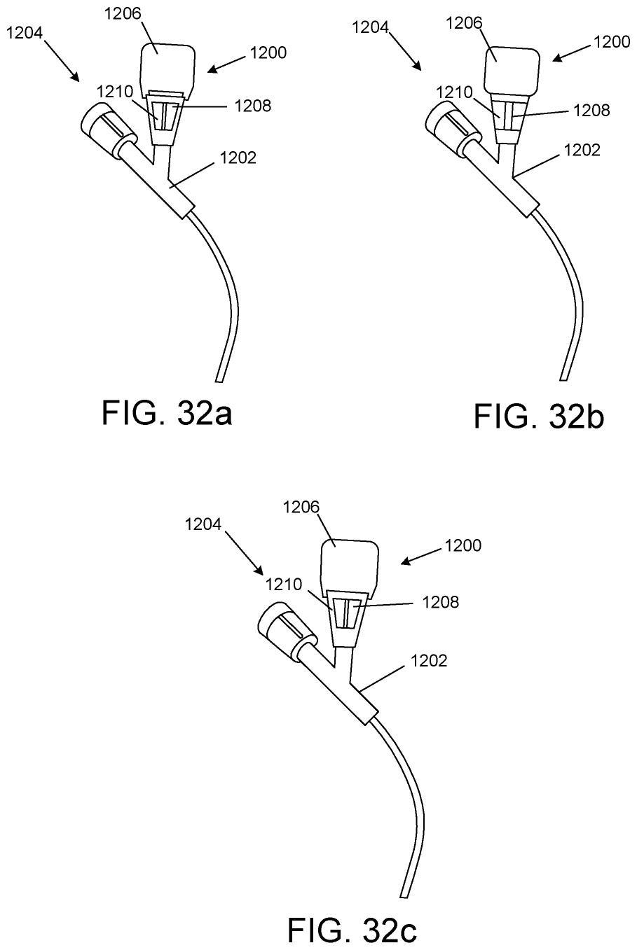

FIGS. 32a to 32c show yet another alternative embodiment of a vent device 1200 in accordance with further aspects of the invention. The vent device 1200 is attached to an adapter 1202 to form part of a port assembly 1204. The engagement body 1206 of the vent device 1200 is movable along the connector 1208 in a distal direction, towards the adapter 1202, to move the valve opener (not shown) located with the engagement body from a non-venting condition shown in FIG. 32a to a venting condition shown in FIG. 32b, to open the valve 1210 inside the connector 1208. Notably, the valve 1210 in FIG. 32a is in a closed condition, whereas the valve 1210 is in an open condition in FIG. 32b.

In an example, the vent device 1200 is advanced from the un-activated condition to the activated condition by rotation, such as by rotating screw threads of the engagement body 1206 with screw threads of the connector 1208. On completion of venting when in the activated condition, the vent device 1200 may be disconnected by counter-rotation in the opposite direction. Alternatively, the vent device 1200 may be rotated back to a non-venting condition as shown in FIG. 32c or FIG. 32a without complete disconnection, or to then disconnected at a later time when appropriate.

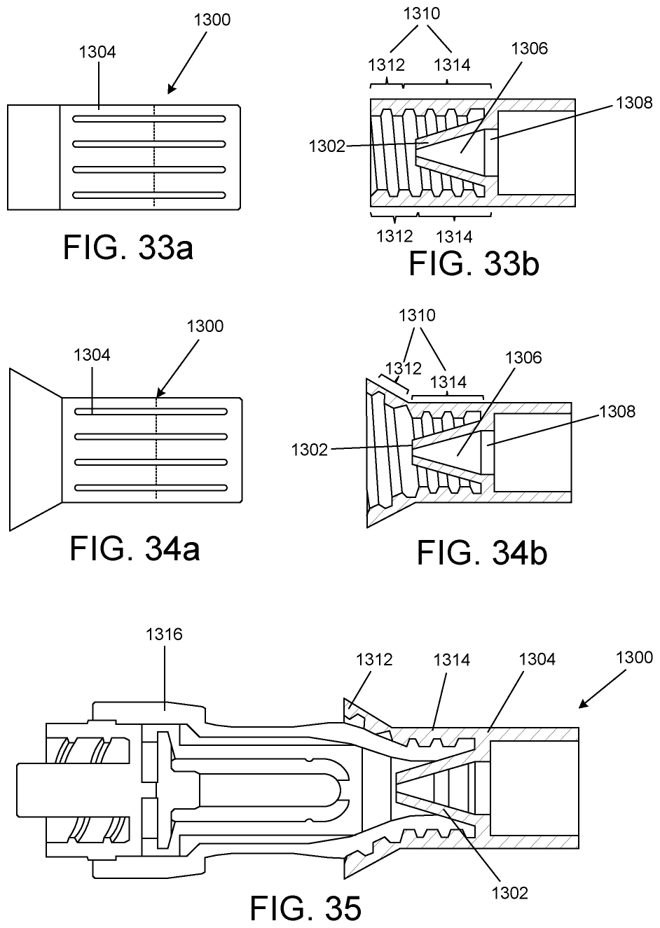

FIGS. 33a to 35 show yet another embodiment of a vent device 1300 in accordance with still further aspects of the invention in various views. Refer initially to the plan view and cross-sectional view of FIGS. 33a and 33b, respectively, the vent device 1300 of the present embodiment comprises a valve opener 1302, an engagement body 1304, and gas-permeable vent 1306 similar to other vent devices discussed elsewhere herein. The present embodiment further includes a semipermeable material 1308, such as a membrane, located within the path of the gas-permeable vent 1306.

The engagement body 1304 comprises an engagement section 1310 having a distal region 1312 and a proximal region 1314. The distal region 1312 is adapted to deform as the valve opener 1302 moves from a non-venting condition shown in FIG. 33b to a venting condition shown in FIG. 34b, which corresponds to the plan or side view of FIG. 34b. In the present embodiment, the distal region 1312 is not symmetrical and is longer to one side of the axis of the vent device 1300 than on the other side. This is shown by the annotated lines 1312, 1312 of FIG. 33b, showing one to be longer than the other. The same different length configuration applies to the proximal region 1314 of the engagement section 1310. In an example, the interface between distal region 1312 and the proximal region 1314 of the engagement section 1310 is a weakening or thinning section, such as a living hinge or change in materials, that follows a helix pattern formed on the inside of the engagement body 1304 for engaging a connector 1316, as shown in FIG. 35.

The distal region 1312 includes slits, perforations and/or other weakening features in the axial direction to enable it to fracture and/or deform as it is rotated or otherwise driven in a distal direction onto the connector 1316. Thus, the engagement body 1304 of the vent device 1300 maintains contact with the connector 1316 in both a non-venting condition and a venting condition of the valve opener 1302. Moreover, due to the plastic deformation of the distal region 1312 of the engagement body 1304, the vent device 1300 cannot be moved back to a non-venting condition since the distal region 1312 has been deformed and will not allow for the transition. Instead, the venting device 1300 must remained engaged to the connector in a venting condition and can only be disconnected after venting without transitioning to a non-venting condition.

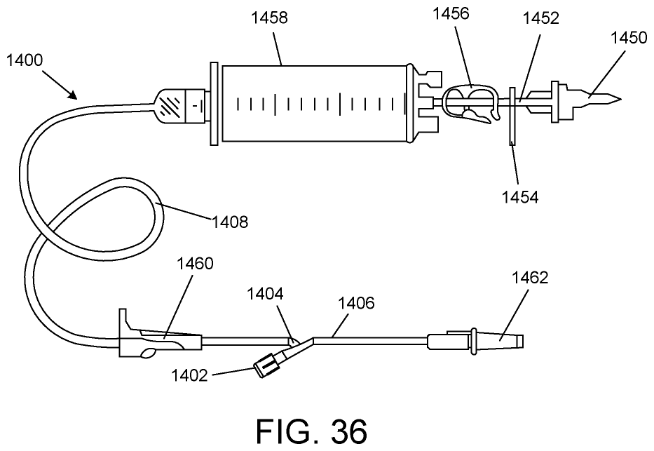

FIG. 36 further illustrates the versatility of the present teachings in expelling or capturing air from a line. In particular, FIG. 36 shows an intravenous (IV) set 1400 comprising a spike 1450 connected to a tubing length 1452, a slide clamp 1452, a tubing clamp 1454, a drip chamber 1456 connected to the other end of the tubing length 1452, a tubing line 1408 connected to the drip chamber outlet, a roller clamp 1458 mounted on the tubing line 1408, a Y-branch or Y-site 1404, which can alternatively be an adapter or hub, a second line 1406 connected downstream of the Y-site, and a Luer adapter 1462 for connecting to a Luer hub, such as a Luer lock collar. In some examples, the IV set can include fewer or additional components. For example, the spike 1450 can be integrated directly with the drip chamber 1458 and more than one adapter or Y-site can be included to provide additional connect points. In some examples, the spike can be integrated with a Y-branch and a one-way vent adapter connector to the Y-branch.

In an example, a needleless valve or port can be connected to the Y-site 1404. The needleless valve or port, which can be called a connector, can then connect to a vent device 1402 to remove air captured or entrained in fluid lines 1406 and 1408, expelled out through the vent device 1402 in the manner discussed above. The vent device 1402 can be one of the vent devices discussed elsewhere herein.

It will be appreciated that many other modifications and permutations of various aspects of the described embodiments are possible. Accordingly, the described aspects are intended to embrace all such alterations, modifications, and variations that fall within the spirit and scope of the appended claims.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Further, while various vent devices, port assemblies, and vascular access assemblies are shown, methods of using and methods of making such devices and assemblies are understood to be within the scope of the invention.

Although limited embodiments of vent devices, port assemblies, and vascular access assemblies and their components have been specifically described and illustrated herein, many modifications and variations will be apparent to those skilled in the art. Accordingly, it is to be understood that the vent devices, port assemblies, and vascular access assemblies and their components constructed according to principles of the disclosed devices, systems, and methods may be embodied other than as specifically described herein. The disclosure is also defined in the following claims.

1. A vent device for venting gas from a connector of an adapter, the connector comprising a valve, the vent device comprising:
a valve opener having a tip;
an engagement body retaining the valve opener; and
a gas-permeable vent formed with the valve opener;
the valve opener is moveable from an initial non-venting condition, in which the valve opener is held in-register with the valve by the engagement body and the tip is not pushed against the valve to open the valve, to a venting condition in which the tip of the valve opener deflects the valve to open the valve and gas passes from within the adapter through the gas-permeable vent of the valve opener.

2. The vent device of claim 1, wherein the gas-permeable vent comprises a semipermeable material.

3. The vent device of claim 1, wherein the valve opener has a body and the gas-permeable vent comprises a flow path formed through the body of the valve opener.

4. The vent device of claim 3, wherein the semipermeable material is located within the flow path.

5. The vent device of claim 1, wherein the engagement body engages the connector in both the initial non-venting condition and the venting condition of the valve opener.

6. The vent device of claim 5, wherein the engagement body has a distal edge that extends distally of a distal end of the valve opener in the initial non-venting condition but not in the venting condition.

7. The vent device of claim 6, wherein the engagement body maintains a gap between the distal end of the valve opener and the valve when the valve opener is in the initial non-venting condition.

8. The vent device of claim 1, wherein the engagement body and the valve opener are axially fixed relative to one another in both the initial non-venting condition and the venting condition of the valve opener.

9. The vent device of claim 5, wherein the engagement body comprises a distal section for engaging the connector, a proximal section for supporting the valve opener, and an intermediate section between the distal section and proximal section, wherein the intermediate section is flexible and deformable so the valve opener can move from the initial non-venting condition to the venting condition when the intermediate section is deformed.

10. The vent device of claim 5, wherein the engagement body is movable along the connector in a distal direction as the valve opener moves from the initial non-venting condition to the venting condition.

11. The vent device of claim 10, wherein the engagement body comprises an engagement section having a distal region and a proximal region, the distal region having a structure that is deformable as the valve opener moves from the initial non-venting condition to the venting condition.

12. The vent device of claim 1, wherein the engagement body comprises at least one arm having a distal tip adapted to engage behind a narrowing in an outer surface of the connector to maintain the valve opener in the venting condition.

13. The vent device of claim 12, wherein the at least one arm comprises a plurality of resilient arms adapted to engage behind the narrowing in the outer surface of the connector, in space relation.

14. The vent device of claim 13, wherein the plurality of resilient arms comprises two arms adapted to engage behind the narrowing in the outer surface of the connector on opposite sides of the connector.

15. The vent device of claim 1, wherein a distal end of the valve opener is slotted.

16. The vent device of claim 1, wherein the engagement body rotates onto the connector to engage the connector, the engagement body comprising one or more finger grip members on an outer surface of the engagement body.

17. A port assembly for a vascular access assembly, comprising:
an adapter comprising at least two connectors; and
a vent device according to claim 1 attached to a first connector of said at least two connectors.

18. The port assembly of claim 17, wherein the adapter is a Y-site.

19. The port assembly of claim 17, wherein the vent device and the first connector define a sterile volume containing a distal end of the valve opener and a proximal end of the valve.

20. A vascular access assembly comprising:
a port assembly according to claim 17;
a catheter assembly; and
an extension tube connecting the port assembly to the catheter assembly.

21. The vascular access assembly of claim 20, further comprising a clamp on the extension tube.

22. A method of venting gas entrapped in a connector comprising:
connecting an engagement body of a vent device to a proximal end of the connector, the connector comprising a valve;
aligning a tip of a valve opener secured to the engagement body in registration with the valve; and
moving the valve opener after connecting the engagement body to the connector from an initial non-venting condition to a venting condition in which the tip pushes against the valve and gas entrapped in the connector flowing out through a gas-permeable vent formed with the valve opener.
